(19) United States
(12) Patent Application Publication
(10) Pub. No.: US 2007/0249058 A1
(43) Pub. Date: Oct. 25, 2007

(54) Hydrocarbon dewpoint measurement device and method

(75) Inventor: Alwarappa Sivaraman, Sugarland, TX (US)

Correspondence Address: MARK E. FEJER;GAS TECHNOLOGY INSTITUTE, 1700 SOUTH MOUNT PROSPECT ROAD, DES PLAINES, IL, 60018, US

(73) Assignee: GAS TECHNOLOGY INSTITUTE

(21) Appl. No.: 11/406,720

(22) Filed: Apr. 19, 2006

(51) Int. Cl. G01N 33/00

(52) U.S. Cl. 436/139

(57) ABSTRACT

A method and system for determining a hydrocarbon dewpoint of natural gas in which a spherical acoustic resonator is filled with natural gas at a first temperature and pressure, where the first temperature is above the hydrocarbon dewpoint at the pressure. An acoustic signal is introduced into the natural gas and the radial resonance frequency of the natural gas is measured as the temperature of the natural gas is reduced. As the temperature decreases, the radial resonance frequency also decreases. The hydrocarbon dewpoint is the temperature at which the radial resonance frequency is at a minimum.

### BACKGROUND OF THE INVENTION

1. Field of the Invention

This invention relates to a method and apparatus for measuring the hydrocarbon dewpoint of natural gas.

2. Description of Related Art

Natural gas is a mixture of gaseous constituents comprising a large portion of methane and lesser amounts of higher straight chain alkanes and various impurities such as carbon dioxide, hydrogen sulphide, saturated water vapor and some heavy-end liquid hydrocarbon compounds. Before the natural gas can be transmitted to the end user, it must first be processed to remove the impurities and reduce excess water and condensable hydrocarbons therein.

Notwithstanding the processing of the natural gas prior to transmission to the end user to remove condensable hydrocarbons, liquid hydrocarbon dropout in natural gas streams which are conveyed through pipelines still has the potential for causing substantial problems for the natural gas pipeline companies in connection with equipment, e.g. compressor malfunctions and metering errors, operational safety, reliability, system integrity, gas quality and environmental issues. Problems in any one of these areas has the potential for interrupting gas transmission through the pipelines and delivery to natural gas customers. As a result, generally strict limitations are imposed on the amount of water permitted in the natural gas streams and on the dewpoint temperature of condensable hydrocarbons present therein, requiring gas suppliers to a pipeline system to monitor and control the hydrocarbon dewpoint temperature at all pipeline inputs.

There are known at present several methods and apparatuses for detecting and monitoring the hydrocarbon dewpoint of natural gas. Dewpoint detectors based upon the principle of detecting the presence of dew on a cooled surface, such as a mirror, represent one such method and apparatus. The mirror is cooled by some means depending upon the lowest mirror temperature desired, and the temperature at which condensation is observed on the mirror is noted as the dewpoint.

Prismatic devices involving visible or infrared light which rely upon the principle of total internal reflection in the absence of a liquid or other medium on the surface are also known. The presence of liquid or other medium on the surface allows light to escape and reduces the intensity of the return beam. Such an imbalance can be used to signal dewpoint when condensed liquid forms on the surface and the change in light intensity can be amplified to drive suitable indicating recorders and relays. See, for example, U.S. Pat. No. 3,528,278.

U.S. Pat. No. 4,946,288 teaches a dewpoint analyzer for independently detecting the dewpoints of both condensable hydrocarbons and water in a gas stream in which changes in the intensity of light scattered from a mirror surface having a polished and highly reflective section and a roughened, less reflective section which is cooled to below the dewpoints of both the condensable hydrocarbons and water are observed. A decrease in the intensity corresponds to the hydrocarbon dewpoint while an increase in the intensity corresponds to the water dewpoint.

Yet another known method for determining the hydrocarbon dewpoint of natural gas is the gas chromatograph.

Unfortunately, these known methods and apparatuses are too expensive to be used in multiple locations for real-time field monitoring.

### SUMMARY OF THE INVENTION

Accordingly, it is one object of this invention to provide a method and apparatus for real-time monitoring of the hydrocarbon dewpoint of natural gas, which is cost-effective and which is suitable for deployment at multiple locations along a natural gas pipeline.

This and other objects of this invention are addressed by a method for determining the hydrocarbon dewpoint of a natural gas stream using a spherical acoustic resonator in which the spherical acoustic resonator is filled with natural gas at a first temperature and pressure, with the first temperature being greater than the hydrocarbon dewpoint at the pressure. An acoustic signal is introduced into the natural gas and the radial resonance frequency of the natural gas is measured as the temperature of the natural gas is reduced to a point at which a first minimum radial resonance frequency is measured. It has been discovered that the reduced temperature at which the first minimum radial resonance frequency is measured corresponds to the hydrocarbon dewpoint.

A system for determining a hydrocarbon dewpoint of natural gas in accordance with one embodiment of this invention comprises a hollow spherical acoustic resonator filled with said natural gas, acoustic means for introducing an acoustic signal into the natural gas, measurement means for measuring a radial resonance frequency of the natural gas, and cooling means for cooling the natural gas. This system is capable of measuring the hydrocarbon dewpoint in real time. In addition, it is fast, accurate, cost-effective, safe and readily deployable at multiple measurement sites. The system is capable of operation at remote locations with a 12V or 24 V DC or solar power source. The system can be used by natural gas providers to manage the impact of pressure reduction and/or ambient temperature variations during gas transmission. The system can also be used in LNG blending to provide quality assurance and telemeter the hydrocarbon dewpoint data by way of a Supervisory Control And Data Acquisition (SCADA) system for control monitoring.

### BRIEF DESCRIPTION OF THE DRAWINGS

These and other objects and features of this invention will be better understood from the following detailed description taken in conjunction with the drawings wherein:

FIG. 1 is a schematic diagram of a system for determining the hydrocarbon dewpoint of natural gas in accordance with one embodiment of this invention; and

FIG. 2 is a diagram showing radial frequency measured as a function of temperature for determining the hydrocarbon dewpoint of natural gas in accordance with one embodiment of this invention.

### DETAILED DESCRIPTION OF THE PRESENTLY PREFERRED EMBODIMENTS

The dewpoint of a gas is the temperature at which a vapor in a space filled with the gas, at a given pressure, will start to condense. The dewpoint of a gas mixture is the temperature, at a given pressure, at which the highest boiling point constituent will start to condense. The hydrocarbon dewpoint of natural gas is the temperature, at a given pressure, at which the highest boiling point hydrocarbon constituent will start to condense.

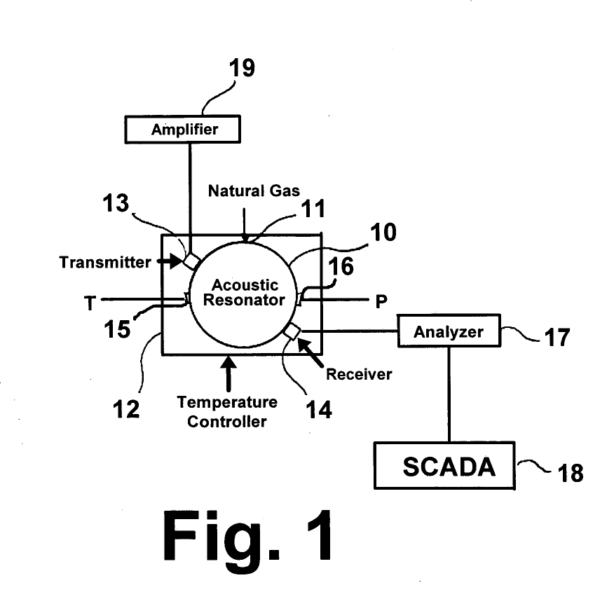

FIG. 1 is a schematic diagram of a system for detecting the hydrocarbon dewpoint of natural gas in accordance with one embodiment of this invention. The system comprises a spherical acoustic resonator 10 having a natural gas inlet 11 through which a natural gas sample is introduced for analysis into the resonator. In accordance with one preferred embodiment of this invention, spherical acoustic resonator 10 is made of stainless steel. Attached to spherical acoustic resonator 10 is an acoustic transmitter 13 for introduction of an acoustic signal into the natural gas sample and an acoustic signal receiver 14 for monitoring the radial resonance frequency of the natural gas sample as the temperature of the natural gas sample is reduced.

The system of this invention further comprises temperature control means for controlling the temperature of the natural gas sample within the spherical acoustic resonator. To provide real-time measurements of the hydrocarbon dewpoint of natural gas, the temperature control means must be capable of quickly reducing the temperature of the natural gas sample, preferably within about 5 minutes. In accordance with one preferred embodiment of this invention, the spherical acoustic resonator is disposed within a thermoelectric cooling system 12, which is capable of varying the temperature of the natural gas sample from about room temperature to temperatures as low as −30° C. using a 12V DC source. Such thermoelectric cooling system components suitable for use in the system of this invention is available from TE Technology, Inc., Traverse City, Mich.

Monitoring of the temperature of the natural gas sample in accordance with one embodiment of this invention is achieved using a temperature sensor 15 in contact with spherical acoustic resonator 10. Contact temperature sensors suitable for use in the system of this invention are well known to those skilled in the art and are readily available.

In addition to temperature, the dewpoint of a gas is also a function of the pressure of the gas. Accordingly, in accordance with one embodiment of this invention, the system of this invention further comprises pressure monitoring means for monitoring the pressure of the natural gas sample within the spherical acoustic resonator. Suitable pressure sensors for use in the system of this invention are also known to those skilled in the art and are readily available.

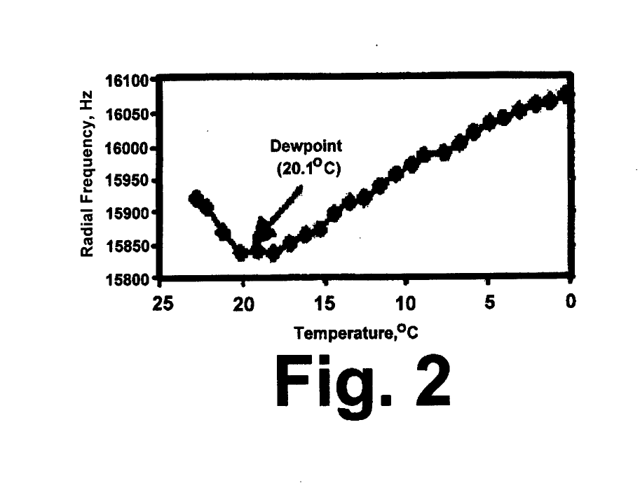

As shown in FIG. 1, the system of this invention further comprises an analyzer 17 operably connected with the signal outputs of acoustic receiver 14, temperature 15 and pressure sensor 16. Analyzer 17, which may be a computer, tracks the temperature and pressure of the natural gas sample together with the radial resonance frequency of the sample as the spherical acoustic resonator is cooled. The temperature at which the first radial resonance frequency occurs is recorded as the hydrocarbon dewpoint. FIG. 2 shows the occurrence of a radial resonance frequency minimum at about 20° C. for a given natural gas sample. At the hydrocarbon dewpoint, condensate droplets begin to form; however, as can be seen, upon nucleation of the droplets, the radial resonance frequency begins to increase due to the gas-liquid phase transition.

As shown in FIG. 1, analyzer 17, in accordance with one embodiment of this invention is operably connected, either wired or wireless, with a SCADA system 18. Data provided by the analyzer to the SCADA system can be used by the natural gas pipeline operator to modify conditions within the pipeline to prevent liquid hydrocarbon dropout within the pipeline.

It will be appreciated by those skilled in the art that the time required to cool a natural gas sample in the system of this invention is affected by the size of the spherical acoustic resonator. Although not critical, it has been found that spherical acoustic resonators having a radius of about 1 inch are suitable for use in the system of this invention.

While in the foregoing specification this invention has been described in relation to certain preferred embodiments thereof, and many details have been set forth for the purpose of illustration, it will be apparent to those skilled in the art that the invention is susceptible to additional embodiments and that certain of the details described herein can be varied considerably without departing from the basic principles of this invention.

What is claimed is:

1. A method for determining a hydrocarbon dewpoint of natural gas comprising the steps of:
filling a spherical acoustic resonator with natural gas at a first temperature and pressure, said first temperature being greater than said hydrocarbon dewpoint at said pressure;
introducing an acoustic signal into said natural gas;
measuring a radial resonance frequency of said natural gas; and
reducing said temperature to a reduced temperature at which a first minimum radial resonance frequency is measured, said reduced temperature corresponding to said hydrocarbon dewpoint.

2. A method in accordance with claim 1, wherein said first temperature is about room temperature.

3. A method in accordance with claim 1, wherein said pressure of said natural gas is measured simultaneously with said temperature.

4. A method in accordance with claim 1, wherein said hydrocarbon dewpoint is reported to a natural gas control system for controlling at least one of natural gas stream composition, natural gas stream temperature, and natural gas stream pressure within a natural gas delivery system.

5. A system for determining a hydrocarbon dewpoint of natural gas comprising:
a hollow spherical acoustic resonator filled with said natural gas;
acoustic means for introducing an acoustic signal into said natural gas;
measurement means for measuring a radial resonance frequency of said natural gas; and
cooling means for cooling said natural gas.

6. A system in accordance with claim 5, wherein said measurement means comprises a radial resonance frequency signal output communicating with a natural gas control system having control means for controlling at least one of natural gas composition, natural gas temperature; and natural gas pressure.

7. A system in accordance with claim 5, wherein said natural gas control system is a Supervisory Control and Data Acquisition (SCADA) system.

8. A system in accordance with claim 6, wherein said communication between said radial resonance frequency signal output and said natural gas control system is wireless.
